(19) United States
(12) Patent Application Publication
(10) Pub. No.: US 2023/0399668 A1
(43) Pub. Date: Dec. 14, 2023

(54) CELL LINES, VARIETIES, AND METHODS FOR IN VITRO COTTON FIBER PRODUCTION

(71) Applicant: Galy Co., Amherst, MA (US)

(72) Inventors: Luciano Luiz Bueno, Boston, MA (US); Paula Maria Elbl, Boston, MA (US); David Dodds, Boston, MA (US); Anat Tewari, Boston, MA (US); Leticia Silveira De Sousa Luz, Divinopolis (BR)

(21) Appl. No.: 18/033,957

(22) PCT Filed: Oct. 26, 2021
(86) PCT No.: PCT/US2021/056600

§ 371 Date: Apr. 26, 2023

Related U.S. Application Data
(60) Provisional application No. 63/231,521, filed on Aug. 10, 2021.
(60) Provisional application No. 63/120,807, filed on Dec. 3, 2020.
(60) Provisional application No. 63/105,767, filed on Oct. 26, 2020.

(51) Int. Cl. C12P 19/04; C12N 5/04; D01C 1/00

(52) U.S. Cl. CPC C12P 19/04; C12N 5/04; D01C 1/00; D10B 2201/02

(57) ABSTRACT

The present disclosure provides in vitro methods for producing cotton fiber, including methods using cotton varietals the were discovered to be particularly amendable for use in the presently disclosed in vitro methods of cotton fiber production.

### BACKGROUND

Cotton is the most widespread non-food crop in the world. However, cotton production is expensive both in terms of money and resources required for its successful cultivation. For example, cotton is a water-intensive crop, with an estimated 9,000-17,000 liters of water required for each kilogram of cotton fiber produced. This equates to enough drinking water to sustain 5,000 people for a day used in order to produce enough cotton to make two t-shirts. Similarly, cotton cultivation requires land, which must be otherwise diverted from other crop production, such as food production. It is estimated that for every acre of cotton grown, only about 500 kilograms of cotton fiber is produced. Cotton cultivation is also a net-emitter of greenhouse gasses, with between approximately 0.75 and 2.25 kilograms of carbon dioxide gas emitted per kilogram of cotton fiber produced. Moreover, because cotton is a plant, its cultivation can lead to failed crops, mistimed crops, and even excess production. Every year, billions of dollars are spent on logistics to overcome unexpected cotton harvest results.

In vitro production of plant cell compositions can overcome a number of limiting factors associated with in planta production of plant-derived products, thereby providing a reliable, energy-efficient, and eco-friendly alternative to traditional agriculture. For example, plant cell compositions produced in vitro can be continuously available, while crops grown in planta are often subject to a cyclic availability.

However, there are no currently known methods for the in vitro production of cotton fiber, especially at an industrial scale. The speed and scale of in vitro production of plant cell compositions currently remain limited by a number of engineering constraints, such as the difficulties of preparing a sufficient amount of cell inoculum of sufficient cellular homogeneity or the lack of streamlined protocols for an in vitro plant cell production cycle.

Accordingly, there is a need for in vitro methods of producing cotton.

### SUMMARY

The present invention provides methods and compositions for the in vitro production of cotton fiber. The methods and compositions of the invention can be scaled up, thereby allowing industrial scale production of cotton fiber. The Inventors have made the surprising discovery that while cells derived from most, if not all, cotton varietals can be used for these in vitro methods of cotton production, certain varietals possessed traits that make them particularly amenable for in vitro cotton fiber production. When used in the in vitro methods of the invention, some of these varietals showed, for example, unexpectedly quick cell growth, fast cell multiplication/duplication, early cotton fiber/pre-fiber growth, and/or efficient bioreactor inoculation.

Further, the Inventors made the surprising discovery that cotton cells from or derived from any meristematic tissue of a cotton plant can be used in the disclosed methods of in vitro cotton fiber production. However, unexpectedly, within certain meristematic tissues, the location on the tissue from which the cells were obtained can have a meaningful impact on cell growth, culture, and fiber development in the presently disclosed in vitro methods of cotton production. For example, the Inventors have surprisingly discovered that cotton ovule cells, including cotton ovule epidermal cells, obtained from different locations on a cotton boll (e.g., the top, middle or bottom third of the boll) provide varying levels of cell growth and fiber development when used in the presently disclosed methods. This variation even occurred when the cells were obtained from cotton plants of the same varietal. The Inventors also made the surprising discovery that the ideal location on a tissue for cell growth, culture and/or fiber development varied among different cotton varietals.

By using the presently disclosed methods of in vitro cotton production, cotton fiber can be produced using approximately 77% less water and 80% less land area than traditional in planta methods. Concurrently, the methods can produce cotton fiber and result in approximately 84% less carbon dioxide emissions when compared with traditional methods. Despite the lower resource costs, the methods of the invention produce cotton fiber much faster that in planta methods. Whereas cotton traditionally requires 5-6 months from planting to harvest, the in vitro methods of the present disclosure can lead to a cotton fiber harvest in approximately 45 days or less. Additionally, because the disclosed methods are in vitro as opposed to inplanta, they can be more rigidly controlled. Therefore, the propensity for failed, mistimed, or excess crops can be reduced, if not completely, eliminated.

Surprisingly, the Inventors of the present invention overcame many of the obstacles associated with in vitro crop production. The Inventors discovered that, unexpectedly, nearly all tissues of a cotton explant can be used to produce a proliferating cell aggregate to inoculate a bioreactor for in vitro cotton production. Further, unexpectedly, cells derived from certain cotton varietals were shown to produce an inoculum, for inoculating a bioreactor, quickly and efficiently. Moreover, the proliferating cell aggregates can be stably cold-stored. Additionally, a bioreactor inoculated with the proliferating cell aggregates can quickly leads to cell doubling, and the doubled cells can be elongated for cotton fiber production.

Thus, the present invention includes a method for producing cotton fiber. In an exemplary method, the method includes inoculating a bioreactor with cotton cells; multiplying the cells in the bioreactor; elongating the multiplied cells; and harvesting cotton fiber from the elongated cells. In certain methods, the cotton cells used to inoculate the bioreactor are from or derived from cotton plants of a varietal selected from PAYMASTER HS26, PD 2164, SA 2413, SEALAND #1 (G.B. X G.H.), SOUTHLAND M1, STATION MILLER, TASHKENT 1, TIDEWATER 29 (G.B. X G.H.), TOOLE, WESTERN STORMPROOF, ACALA 5, ALLEN 33, CD3HCABCUH-1-89, DELTAPINE 14, DES 24, DIXIE KING, FJA, M.U.8B UA 7-44, NC 88-95, PAYMASTER HS200, Pima S-7, Acala and MAXXA, or a progeny of any thereof. In certain methods, the varietal is selected from PD2164, WESTERN STORMPROOF, CD3HCABCUH-1-89, TASHKENT 1, SOUTHLAND M1, ACALA 5, FJA, PAYMASTER HS200, Pima S-7, and Acala MAXXA, or a progeny of any thereof. In some methods, the varietal is selected from PD 2164, SOUTHLAND M1, ACALA 5, CD3HCABCUH-1-89, FJA, Pima S-7, and Acala MAXXA, or a progeny of any thereof. In certain methods, the varietal is selected from PD 2164, SOUTHLAND M1, and CD3HCABCUH-1-89, or a progeny of any thereof. Further, in certain methods, the varietal is PD 2164.

In certain methods, the methods include inoculating the bioreactor with cotton ovule cells, which may include cotton ovule epidermal cells. The cotton ovule and ovule epidermal cells may be obtained from a cotton boll. In certain methods, the cotton ovule and/or ovule epidermal cells are obtained from a bottom third, a middle third, or a top third of the cotton boll, wherein the bottom is a location on the boll to which its growth from a cotton plant stem began. In certain methods, the cotton ovule and/or ovule epidermal cells are obtained from the top third of the cotton boll, and the varietal is selected from PD 2164 and ACALA 5, or a progeny of any thereof. In certain methods, the cotton ovule and/or ovule epidermal cells are obtained from the middle third of the cotton boll, and the varietal is selected from PD 2164 and FJA, or a progeny of any thereof. In certain methods, the cotton ovule and/or ovule epidermal cells are obtained from the bottom third of the cotton boll, and the varietal is selected from PD 2164, SOUTHLAND M1, ACALA 5, CD3HCABCUH-1-89, FJA, Pima S-7, and Acala MAXXA, or a progeny of any thereof.

The methods for producing cotton fiber may include inoculating the bioreactor with cotton ovule cells. The methods may alternatively or additionally include inoculating the bioreactor with cells from a proliferating cell aggregate. In certain methods, the cells of the proliferating cell aggregate are obtained and/or derived from a cotton plant of the varietal Pima S-7.

Preferably, the proliferating cell aggregate is a friable callus. Thus, the methods may further include obtaining cells from a cotton explant; and contacting the cells from the cotton explant with a callus induction medium to produce the friable callus. Surprisingly, the cells from a cotton explant are from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

The methods may further include dissociating cells from the friable callus; and culturing the dissociated cells. The cultured dissociated cells may be used to inoculate the bioreactor. Culturing the dissociated cells may include culturing the dissociated cells in a liquid or semi-solid medium to form a cell suspension. The methods may also include cryopreserving the cell suspension; and inoculating the bioreactor with the cryopreserved cell suspension. the method further comprises homogenizing the cell suspension to form a fine cell suspension. Homogenizing may include one or more of subculturing the cell suspension; filtering the cell suspension; pipetting and/or decanting the cell suspension; and adding pectinase to the suspension.

The methods may also include separating the elongated cells from any non-elongated cells; and harvesting cotton fiber from the separated elongated cells. In addition, the method may include recycling any non-elongated cells for use in subsequent iterations of the method.

The methods for producing cotton may also include maturing the elongated multiplied cells using a maturation medium; and harvesting cotton fiber from the mature elongated cells. Harvesting may include separating cotton fiber from the maturation medium; and passing air through the separated cotton fibers until the fibers have a moisture content of less than 5%.

In certain aspects, the methods for producing cotton can include inoculating the bioreactor with cotton cells from and/or derived from a cotton plant of the Gossypium species selected from G. hirsutum, G. arboreum, G. barbadense, G. anomalum, G. armourianum, G. klotzchianum, G. raimondii, G. herbaceum, or a progeny of any thereof. In certain aspects, the cotton plant is of a variety selected from PAYMASTER HS26, PD 2164, SA 2413, SEALAND #1 (G.B. X G.H.), SOUTHLAND M1, STATION MILLER, TASHKENT 1, TIDEWATER 29 (G.B. X G.H.), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, or a progeny of any thereof.

In certain aspects, the methods of the present invention can be used to produce at least 1 kilogram of cotton fiber for every 4,000 liters of water used in the method. In some instances, the methods of the invention can produce at least 1 kilogram of cotton fiber for between every 2,000 and 4,000 of water used in the method.

In certain aspects, the present invention includes an in vitro method for producing cotton fiber using cells obtained and/or derived from a cotton plant of a varietal selected from PD2164, WESTERN STORMPROOF, CD3HCABCUH-1-89, TASHKENT 1, SOUTHLAND M1, ACALA 5, FJA, PAYMASTER HS200, Pima S-7, and Acala MAXXA, or a progeny of any thereof.

In certain aspects, the present invention includes an in vitro method for producing cotton fiber using cells obtained and/or derived from a cotton plant of a varietal selected from PD2164, SOUTHLAND M1, FJA, PAYMASTER HS200, TIDEWATER 29, TASHKENT 1, DIXIE KING, and Acala MAXXA, or a progeny of any thereof.

### BRIEF DESCRIPTION OF THE DRAWINGS

FIG. 1 shows an exemplary method of the invention

FIG. 2 shows a flowchart of the concept of a commercial scale process for the cotton fiber in vitro production.

FIG. 3 shows a computer system that is programmed or otherwise configured to implement methods provided herein.

### DETAILED DESCRIPTION

The present invention provides methods and compositions for the in vitro production of cotton fiber. The methods of the disclosure can be cell-based, and not require the growth of entire cotton plants for the cultivation of cotton fiber. These methods allow quick and efficient cultivation of cotton fiber in a controlled environment.

Surprisingly, while cells derived from most, if not all, cotton varietals can be used for these in vitro methods of cotton production, some varietals possess traits that make them particularly useful for the presently disclosed methods of in vitro cotton fiber production. When used in the methods of the invention, some of these varietals show, for example, unexpectedly quick cell growth, fast cell multiplication/duplication, early cotton fiber/pre-fiber growth, and/or efficient bioreactor inoculation.

Further, the Inventors made the unexpected discovery that cotton cells from and/or derived from any meristematic tissue of a cotton plant can be used in the disclosed methods of in vitro cotton fiber production. Though, surprisingly, within certain meristematic tissues, the location on the tissue from which the cells were obtained can impact cell growth, culture, and fiber development in the presently disclosed in vitro methods of cotton production. For example, the Inventors discovered that cotton ovule and/or ovule epidermal cells obtained from different locations on a cotton boll (e.g., the top, middle or bottom third of the boll) provide varying levels of cell growth and fiber development when used in the presently disclosed methods. This variation occurred even when the cells were obtained from cotton plants of the same varietals. The Inventors also made the surprising discovery that the ideal location on a tissue for cell growth, culture and/or fiber development was different among different cotton varietals.

Advantageously, the methods and compositions of the invention can be scaled up, thereby allowing industrial scale production of cotton fiber. By using these methods of in vitro cotton production, cotton fiber can be produced using approximately 77% less water and 80% less land than traditional in planta methods. The methods can also produce cotton fiber harvest that results in about 84% less carbon dioxide emissions when compared with traditional methods. Despite the lower resource costs, the methods of the invention produce cotton fiber much faster that in planta methods. Whereas cotton traditionally requires 5-6 months from planting to harvest, the in vitro methods of the present disclosure can lead to a cotton fiber harvest in approximately 45 days.

Additionally, because the disclosed methods are in vitro as opposed to in planta, they can be more rigidly controlled. Therefore, the propensity for failed, mistimed, or excess crops can be reduced, if not completely, eliminated. These methods can be practiced indoors, using automated machinery, and even specialized cotton cell lines to ensure a cotton fiber harvest with desired qualities.

Surprisingly, the Inventors of the present invention overcame many of the obstacles associated with in vitro crop production. The Inventors discovered that, unexpectedly, nearly all tissues of a cotton explant can be used to produce a proliferating cell aggregate to inoculate a bioreactor for in vitro cotton production. Moreover, the proliferating cell aggregates can be stably cold-stored. Thus, once created, cell aggregates can be used for cotton fiber production without the need to rely on living cotton plants. A bioreactor inoculated with a small number of cells from the proliferating cell aggregate quickly leads to cell doubling in the bioreactor, and the doubled cells can be elongated for cotton fiber production.

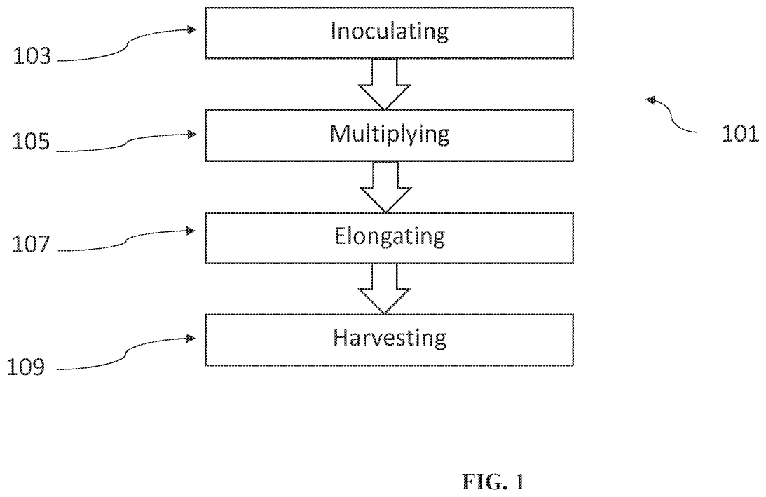

FIG. 1 provides an exemplary method 101 of the disclosure. First a bioreactor is inoculated 103 with a small number of cotton cells. The bioreactor may be inoculated with a small number of cotton ovule cells, which may include ovule epidermal cells. Generally, the bioreactor will be inoculated with a small number of cotton cells from a proliferating cell aggregate. As shown in Example 4, milligram quantities of cotton cells from a proliferating cell aggregate are sufficient to eventually inoculate a bioreactor.

Inoculating 103 may include preparing a growth medium in a vessel, such as a flask or plate, and introducing a small number of cotton cells from a proliferating cell aggregate into the medium. The vessel may then be left for inoculum growth. Alternatively, inoculum growth may occur inside the bioreactor.

The Inventors found that, surprisingly, inoculum growth under dark conditions provided superior growth. The vessel may be shaken or agitated during inoculum growth, for example, at a rate of 80-180 rpm. Preferably, inoculum growth occurs at a temperature of about 30° C. to about 35° C. Preferably, the medium is a solution that comprises plant hormones, plant growth regulators, and/or sucrose and/or glucose. Inoculum growth generally takes about 16 days, but may be more or less as desired or due to conditions or individual cotton cell lines.

The inoculum may be a cell suspension in a liquid or semi-solid medium. The suspension may be optionally homogenized to provide a fine cell suspension culture. The present Inventors discovered that a homogenous cell suspension can provide more reproducible and reliable results when inoculating a bioreactor.

Homogenizing may include any methods known in the art, including one or more of subculturing the suspension, filtering, pipetting/decanting, and/or addition of a low concentration of pectinase.

The resulting inoculum is then introduced into a bioreactor. Alternatively, the resulting inoculum can be preserved, e.g., by freezing, for later use in inoculating a bioreactor. The inoculum or homogenous cell suspension may be cryopreserved indefinitely, for example, in liquid nitrogen. This generally requires suspending cells from the inoculum/homogenous cell suspension in a cryoprotectant solution, for example a solution of glycerol and sucrose. The cryoprotectant solution can be supplement, for example, using proline. Cryopreserved cells can be recovered, for example, using a recovery media, before their use in inoculating a bioreactor.

The proliferating cell aggregate may be a callus. Preferably, the proliferating cell aggregate is a friable callus, which is not sticky or soft, but is also not so hard or dense that it cannot be physically broken or crumbled. A friable callus thus differs “a hard callus”, which is compact and brittle, and thus not amenable to being broken or crumbled. The Inventors discovered that a friable callus allows for simple mechanical manipulation to easily disassociate individual cells from the friable callus for use in inoculating 103 a bioreactor and/or preparing an inoculum.

After inoculating 103, the method 101 requires multiplying 105 the cells in the bioreactor. This phase generally lasts for between 5 and 12 days, with duplication for the cotton cells taking approximately 1 to 3 days depending on cotton lineage. The cells may be duplicated, for example, by culturing the cells in a cell culture medium.

The multiplied cells are then elongated 107 to produce cotton fibers. This may include using an elongation medium to induce elongation in the multiplied cells. In certain aspects, the elongation medium facilitates release of a phenolic compound from a vacuole of an elongated cotton cell. The elongated cells may include cotton pre-fibers, which will mature into cotton fibers.

In certain aspects, a semi-solid elongation medium is used to elongate the cotton cells. The Inventors made the surprising discovery that superior results are achieved when using a semi-solid medium as opposed to a liquid medium.

Optionally, after elongation, the elongated cotton cells are separated from any non-elongated cotton cells. The non-elongated cotton cells will not mature into cotton fibers. However, they may be recycled and used in subsequent iterations of the method. Separating the elongated cotton cells from the non-elongated cells may include one or more of filtering, sieving, decanting, and centrifuging the cells.

Once separated, the elongated cotton cells, which at this point may have cotton pre-fibers, are matured. Maturing the cells may include the use of a maturation medium. During maturation, sugars are combined in the cells to produce cellulose, which is the main component of cotton fiber (natural glucose polymerization) that occurs inside the cell forming a secondary wall. The cotton pre-fibers increase in number, density, and/or length.

After maturation, cotton fiber harvested from the cotton cells, by for example, separating the fibers from the cells in a solution/buffer. The harvested cotton fiber is then dried to a moisture content of less than 5% by, for example, passing air through the cotton fiber.

Thus, the method 101 can produce cotton fibers from cotton cells without growing cotton plants. These methods allow quick and efficient cultivation of cotton fiber in a controlled environment.

The method 101 may also include preparing a friable callus. A friable callus can be made, for example, by obtaining cells from a cotton explant and contacting the cells with a callus induction medium. Surprisingly, the Inventors discovered that tissue from any meristematic part of a cotton plant can be used to produce a friable callus. Thus, the cells from the cotton explant can from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

Preparing a friable callus may include contacting the cells of a cotton explant with a callus induction medium. The callus induction medium may facilitate the division of at least a subset of cells of a plant explant. Using the callus induction medium results in dedifferentiated cell masses. The cells in these masses can be subsequently cultured, which may include the use of a callus growth medium.

Certain aspects of the invention require the use of plant hormone(s) and/or growth regulator(s) (including auxins, gibberellins, etc.). The hormones/regulators can be used, for example, in the mediums described herein for culturing cotton cells. Plant hormones and/or growth regulators (including auxins, gibberellins, etc.) can be derived from naturally occurring sources, synthetically produced, or semi-synthetically produced, i.e., starting from naturally derived starting materials then synthetically modifying said materials. These modifications can be conducted using conventional methods as envisioned by a skilled worker. The following references include plant hormones and/or growth regulators (including auxins, gibberellins, etc.) for plant cell composition as described hereinbelow or described anywhere else herein: Gaspar et al. In Vitro Cell. Dev. Biol-Plant, 32, 272-289, October-December 1996 and Zhang et al. Journal of Integrative Agriculture, 2017, 16(8): 1720-1729; the contents of each of which (particularly, all the plant hormones and/or plant growth regulators) are incorporated by reference herein. In particular, one of skill in the art will understand that certain gibberellins are capable of facilitating plant cell elongation.

In some aspects, plant hormones and/or growth regulators used in the present invention are exemplified by those in Table A.

TABLE A
Exemplary plant hormones or plant growth regulators
and exemplary applications in plant cell engineering.

| | | | | Fiber | | |
|---|---|---|---|---|---|---|
| | | Callus | | initiation/ | Cell wall | Other |
| Name | Abbreviation | induction | Multiplication | elongation | thickening | applications |
| indole acetic acid | IAA | Y | Y | Y | Y | |
| indole butyric acid | IBA | Y | Y | Y | Y | |
| 2,4- | 2,4 D | Y | Y | Y | Y | |
| dichlorophenoxyacetic | | | | | | |
| acid | | | | | | |
| naphthaleneacetic acid | NAA | Y | Y | Y | Y | |
| para-chlorophenoxyacetic | pCPA | Y | Y | Y | Y | |
| acid | | | | | | |
| β-naphthoxyacetic acid | NOA | Y | Y | Y | Y | |
| 2-benzothiazole acetic | BTOA | Y | Y | Y | Y | |
| acid | | | | | | |
| picloram | PIC | Y | Y | Y | Y | |
| 2,4,5,- | 2,4,5-T | Y | Y | Y | Y | |
| trichlorophenoxyacetic | | | | | | |
| acid | | | | | | |
| phenylacetic acid | PAA | Y | Y | Y | Y | |
| kinetin | KIN | Y | Y | Inhibitor | ND | |
| 6-benzylaminopurine | 6BA | Y | Y | Inhibitor | ND | |
| N6-(2-isopentenyl) | 2iP | Y | Y | Inhibitor | ND | |
| adenine | | | | | | |
| zeatin | ZEA | Y | Y | Inhibitor | ND | |
| gibberellin A1 | GA1 | ND | ND | Y | ND | Control fiber |
| gibberellic acid | GA3 | ND | ND | Y | ND | |
| gibberellin A4 | GA4 | ND | ND | Y | ND | strength, |
| gibberellin A7 | GA7 | ND | ND | Y | ND | micronaire and |
| | | | | | | maturation |
| ethylene | — | ND | ND | Y | ND | |
| brassinolide | BR | ND | ND | Y | Y | |
| jasmonic acid | JA | ND | ND | Y | ND | |

“Y” indicates that the corresponding plant hormone or plant growth regulator in the row can be used for the application indicated in the column heading.
“Inhibitor” indicates that the corresponding plant hormone or plant growth regulator in the row can be used for inhibiting the activity indicated in the column heading.
“ND” indicates that effect(s) of the corresponding plant hormone or plant growth regulator for the application indicated in the column heading is not yet determined (at least to some extent).

In certain aspects, the invention uses an induction medium or callus induction medium. The callus induction medium described herein can be configured to facilitate division of at least a subset of cells of a plant explant. For example, the callus induction medium can facilitate or promote induction of a cotton plant callus. The callus induction medium can comprise a diluted basal medium (i.e., from 1:1.5 to 1:5, from 1:1.5 to 1:4, from 1:1.5 to 1:3, etc.). The callus induction medium can comprise one or more salts, macronutrients, micronutrients, organic molecules, and/or hormones (such as those that can facilitate or promote induction). The callus induction medium can be a liquid at about 25° C. Alternatively, the callus induction medium can be not a liquid at a specified temperature. In some embodiments, the callus induction medium is not a liquid at about 25° C. In some embodiments, the callus induction medium can be a semi-solid medium (such as gelled) at 25° C.

Non-limiting examples of a semi-solid medium include soft agar, soft agarose, soft methylcellulose, xanthan gum, gellan gum, carrageenan, isabgol, guar gum, other soft polymeric gels, or any other gelling agent known in the art. The callus induction medium can comprise agar. In some embodiments, the callus induction medium can be agar-free. In some embodiments, the callus induction medium is free of any gelling agent. In some embodiments, the callus induction medium that is agar- or gelling agent-free can be a liquid. In some embodiments, the callus induction medium that is agar- or gelling agent-free can be a solid. In some embodiments, the callus induction medium that is agar-free can be a gel. In some embodiments, the callus induction medium that is agar-free can comprise an agar-substitute. In some embodiments, the callus induction medium can have a pH. The pH of the callus induction medium can be appropriate for induction of a plant callus. In some embodiments, the pH of the callus induction medium can be optimized for induction of a plant callus. In some embodiments, the pH of the callus induction medium can be from 5.3 to 6.3. In some embodiments, the pH of the callus induction medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present disclosure includes callus mediums or callus growth mediums, and their use in the in vitro methods for producing cotton. The callus growth medium described herein can facilitate or promote growth of a plant callus and/or produce a proliferating cell aggregate. The callus growth medium can be a gel medium, and in some embodiments, can comprise agar and/or another gelling agent and a mixture of macronutrients and micronutrients for the plant type of the plant callus. In some cases, the callus medium can be enriched with nitrogen, phosphorus, or potassium. In some cases, a callus growth medium can be a liquid medium. In some embodiments, the callus growth medium can comprise at least one plant hormone or growth regulator (including auxins, gibberellins, etc.), or at least two plant hormones or growth regulators, or at least three plant hormones or growth regulators, or at least four plant hormones or growth regulators, or at least five plant hormones or growth regulators, or at least six plant hormones or growth regulators, or at least seven plant hormones or growth regulators, or at least eight plant hormones or growth regulators. The at least one plant hormone or plant growth regulator (or at least two, at least three, at least four, at least five, or at least six plant hormones or plant growth regulators) (including auxins, gibberellins, etc.) can be any one or combination selected from the group consisting of indole acetic acid (IAA), Indoyl-3-acrylic acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, indole butyric acid (IBA), 2,4-dichlorophenoxyacetic acid (2,4 D), tryptophan, phenylacetic acid (PAA), Glucobrassicin, naphthaleneacetic acid (NAA), picloram (PIC), Dicamba, ethylene, para-chlorophenoxyacetic acid (pCPA), β-naphthoxyacetic acid (NOA), benzo(b)selenienyl-3 acetic acid, 2-benzothiazole acetic acid (BTOA), N6-(2-isopentenyl) adenine (2iP), zeatin (ZEA), dihydro-Zeatin, Zeatin riboside, kinetin (KIN), 6-(benzyladenine)-9-(2-tetrahydropyranyl)-9H-purine, 2,4,5-trichlorophenoxyacetic acid (2,4,5-T), 6-benzylaminopurine (6BA), 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, gibberellin A₅, gibberellin A1 (GA1), gibberellic acid (GA3), gibberellin A4 (GA4), gibberellin A7 (GA7), brassinolide (BR), jasmonic acid (JA), gibberellin A₈, gibberellin A₃₂, gibberellin A₉, 15-β-OH-gibberellin A₃, 15-β-OH-gibberellin A₅, 12-β-OH-gibberellin A₅, 12-α-gibberellin A₅, salicylic acid, (−)jasmonic acid, (+)-7-iso-jasmonic acid, putrescine, spermidine, spermine, oligosaccharins, and stigmasterol. The at least one plant hormone or plant growth regulator (or at least two, at least three, at least four, at least five, or at least six plant hormones or plant growth regulators) (including auxins, gibberellins, etc.) can be any one or combination selected from the group consisting of indoyl-3-acetic acid, indoyl-3-acrylic acid, indoyl-3-butyric acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, tryptophan, phenylacetic acid, Glucobrassicin, 2,4-Dichlorophenyoxyacetic acid, 1-naphthaleneacetic acid, Dicamba, Pichloram, ethylene, benzo(b)selenienyl-3 acetic acid, trans-Zeatin, N⁶-(2-isopentyl)adenine, dihydro-Zeatin, Zeatin riboside, Kinetin, benzylamide, 6-(benzyladenine)-9-(2-tetrahydropyranyl)-9H-purine, 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, Gibberellin A₁, Gibberellin A₃, Gibberellin A₄, Gibberellin A₅, Gibberellin A₇, Gibberellin A₈, Gibberellin A₃₂, Gibberellin A₉, 15-β-OH Gibberellin A₃, 15-β-OH Gibberellin A₅, 12-β-OH Gibberellin A₅, 12-α-Gibberellin A₅, salicylic acid, jasmonic acid, (−) jasmonic acid, (+)-7-iso-jasmonicacid, putrescine, spermidine, spermine, oligosaccharins, brassinolide, and stigmasterol.

The at least one plant hormone or plant growth regulator (or at least two, at least three, at least four, at least five, or at least six plant hormones or plant growth regulators) (including auxins, gibberellins, etc.) can be any one or combination selected from the group consisting of indole acetic acid (IAA), indole butyric acid (IBA), 2,4-dichlorophenoxyacetic acid (2,4 D), naphthaleneacetic acid (NAA), para-chlorophenoxyacetic acid (pCPA), 0-naphthoxyacetic acid (NOA), 2-benzothiazole acetic acid (BTOA), picloram (PIC), 2,4,5-trichlorophenoxyacetic acid (2,4,5-T), phenylacetic acid (PAA), kinetin (KIN), 6-benzylaminopurine (6BA), N6-(2-isopentenyl) adenine (2iP), zeatin (ZEA), gibberellin A1 (GA1), gibberellic acid (GA3), gibberellin A4 (GA4), gibberellin A7 (GA7), ethylene, brassinolide (BR), and jasmonic acid (JA).

In certain aspects, the callus growth medium can be a liquid at about 25° C. In some embodiments, the callus growth medium can be not a liquid at about 25° C. In some embodiments, the callus growth medium can be a semi-solid medium (such as gelled) at 25° C. Non-limiting examples of a semi-solid medium include soft agar, soft agarose, soft methylcellulose, xanthan gum, gellan gum, carrageenan, isabgol, guar gum, other soft polymeric gels, or any other gelling agent known in the art. In some embodiments, the callus growth medium can comprise agar. In some embodiments, the callus growth medium can be agar-free. In some embodiments, the callus growth medium is free of any gelling agent. In some embodiments, the callus growth medium that is agar- or gelling agent-free can be a liquid. In some embodiments, the callus growth medium that is agar- or gelling agent-free can be a solid. In some embodiments, the callus growth medium that is agar-free can be a gel. In some embodiments, the callus growth medium that is agar-free can comprise an agar-substitute.

In some embodiments, the callus growth medium can have a pH. The pH of the callus growth medium can be appropriate for growing a plant callus and/or producing a proliferating cell aggregate. In some embodiments, the pH of the callus growth medium can be optimized for growing a plant callus and/or producing a proliferating cell aggregate. In some embodiments, the pH of the callus growth medium can be from 5.3 to 6.3. In some embodiments, the pH of the callus growth medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present invention includes cell culture mediums (e.g., a multiplication/duplication mediums), and their use in the in vitro methods for producing cotton described herein. In some embodiments, the cell culture medium described herein can facilitate or promote proliferation of a cell population, or a proliferating cell aggregate. The cell culture medium can comprise one or more salts, macronutrients, micronutrients, organic molecules, and/or hormones (such as those that can facilitate or promote proliferation). In some cases, the cell culture medium can be configured to proliferate a cell population, such as a proliferating cell aggregate. The cell culture medium can comprise an enzyme that can degrade a plant cell wall of a plant cell of a cell population, or a proliferating cell aggregate. In some embodiments, the enzyme can be a pectocellulolytic enzyme. In some embodiments, the enzyme can comprise cellulase, hemicellulose, cellulysin, or a combination thereof. In some embodiments, the cell culture medium can have a pH. The pH of the cell culture medium can be appropriate for culturing a cell population, or a proliferating cell aggregate.

In some embodiments, the pH of the cell culture medium can be optimized for culturing a cell population, such as a proliferating cell aggregate. In some embodiments, the pH of the cell culture medium can be optimized for cell division. In some embodiments, the pH of the cell culture medium can be from 5.3 to 6.3. In some embodiments, the pH of the cell culture medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values. In some embodiments, the cell culture medium can have a different pH than a callus growth medium. In some embodiments, the cell culture medium can have a same pH as a callus growth medium. In some embodiments, the pH of the cell culture medium can differ from a pH of a callus growth medium by less than 0.1, less than 0.2, or less than 0.3 units. For example, the pH of a cell culture medium can differ from a pH of a callus growth medium by less than 0.2 units.

Preferably, a cell culture medium of the present disclosure includes one or more of MS, B5, glucose, sucrose, Kinetin, 2,4-dichlorophenoxyacetic acid (2,4-D), NAA, and coconut water. Preferably, the cell culture medium comprises 2,4-D. In certain aspects, the cell culture medium includes MS, B5, glucose/sucrose, and 2,4-D.

The present invention also includes recovery mediums, and their use in the in vitro methods for producing cotton fiber. A recovery medium can be used, for example, for recovery of cotton cell inoculum after cryopreservation. Some embodiments described herein are related to a recovery medium. In some embodiments, the recovery medium described herein can be a medium that can facilitate or promote recovery of cotton cells. The recovery medium can comprise one or more salts, macronutrients, micronutrients, organic molecules, and/or hormones that can facilitate or promote elongation.

The present invention includes elongation mediums, and their use in the in vitro methods for producing cotton fiber. The elongation mediums described herein can facilitate or promote elongation of cells capable of being elongated, for example, elongation of cotton cells. The elongation mediums described herein can comprise one or more salts, macronutrients, micronutrients, organic molecules, and/or hormones (such as those that can facilitate or promote elongation). In some embodiments, the elongation mediums can be configured to facilitate a release of a phenolic compound from a vacuole from a cotton cell. In some embodiments, the phenolic compound (such as O-diphenol) is configured to initiate fiber differentiation by inhibiting indoleacetic acid (IAA) oxidase and/or increase an intracellular auxin level. In some embodiments, the elongation medium can comprise at least one plant hormone or growth regulator (including auxins, gibberellins, etc.), or at least two plant hormones or growth regulators, or at least three plant hormones or growth regulators, or at least four plant hormones or growth regulators, or at least five plant hormones or growth regulators, or at least six plant hormones or growth regulators, or at least seven plant hormones or growth regulators, or at least eight plant hormones or growth regulators. The at least one plant hormone or plant growth regulator (or at least two, at least three, at least four, at least five, or at least six plant hormones or plant growth regulators) (including auxins, gibberellins, etc.) can be any one or combination selected from the group consisting of indole acetic acid (IAA), Indoyl-3-acrylic acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, indole butyric acid (IBA), 2,4-dichlorophenoxyacetic acid (2,4 D), tryptophan, phenylacetic acid (PAA), Glucobrassicin, naphthaleneacetic acid (NAA), picloram (PIC), Dicamba, ethylene, parachlorophenoxyacetic acid (pCPA), 0-naphthoxyacetic acid (NOA), benzo(b)selenienyl-3 acetic acid, 2-benzothiazole acetic acid (BTOA), N6-(2-isopentenyl) adenine (2iP), zeatin (ZEA), dihydro-Zeatin, Zeatin riboside, kinetin (KIN), 6-(benzyladenine)-9-(2-tetrahydropyranyl)-9H-purine, 2,4,5-trichlorophenoxyacetic acid (2,4,5-T), 6-benzylaminopurine (6BA), 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, gibberellin A₅, gibberellin A1 (GA1), gibberellic acid (GA3), gibberellin A4 (GA4), gibberellin A7 (GA7), brassinolide (BR), jasmonic acid (JA), gibberellin A₈, gibberellin A₃₂, gibberellin A₉, 15-β-OH-gibberellin A₃, 15-β-OH-gibberellin A₅, 12-β-OH-gibberellin A₅, 12-α-gibberellin A₅, salicylic acid, (−) jasmonic acid, (+)-7-isojasmonic acid, putrescine, spermidine, spermine, oligosaccharins, and stigmasterol. The at least one plant hormone or plant growth regulator (or at least two, at least three, at least four, at least five, or at least six plant hormones or plant growth regulators) (including auxins, gibberellins, etc.) can be any one or combination selected from the group consisting of indoyl-3-acetic acid, indoyl-3-acrylic acid, indoyl-3-butyric acid, 4-Cl-Indoyl-3-acetic acid, Indoyl-3-acetylaspartate, indole-3-acetaldehyde, indole-3-acetonitrile, indole-3-lactic acid, indole-3-propionic acid, indole-3-pyruvic acid, tryptophan, phenylacetic acid, Glucobrassicin, 2,4-Dichlorophenyoxyacetic acid, 1-naphthaleneacetic acid, Dicamba, Pichloram, ethylene, benzo(b)selenienyl-3 acetic acid, trans-Zeatin, N⁶-(2-isopentyl)adenine, dihydro-Zeatin, Zeatin riboside, Kinetin, benzylamide, 6-(benzyladenine)-9-(2-tetrahydropyranyl)-9H-purine, 1,3-diphenylurea, N-(2-chloro-4-pyridyl)-N′-phenylurea, (2,6-dichloro-4-pyridyl)-N′-phenylurea, N-phenyl-N′-1,2,3-thiadiazol-5-ylurea, Gibberellin A₁, Gibberellin A₃, Gibberellin A₄, Gibberellin A₅, Gibberellin A₇, Gibberellin A₈, Gibberellin A₃₂, Gibberellin A₉, 15-β-OH-Gibberellin A₃, 15-β-OH-Gibberellin A₅, 12-β-OH-Gibberellin A₅, 12-α-Gibberellin A₅, salicylic acid, jasmonic acid, (−) jasmonic acid, (+)-7-iso-jasmonicacid, putrescine, spermidine, spermine, oligosaccharins, brassinolide, and stigmasterol.

The at least one plant hormone or plant growth regulator (or at least two, at least three, at least four, at least five, or at least six plant hormones or plant growth regulators) (including auxins, gibberellins, etc.) can be any one or combination selected from the group consisting of indole acetic acid (IAA), indole butyric acid (IBA), 2,4-dichlorophenoxyacetic acid (2,4 D), naphthaleneacetic acid (NAA), para-chlorophenoxyacetic acid (pCPA), 0-naphthoxyacetic acid (NOA), 2-benzothiazole acetic acid (BTOA), picloram (PIC), 2,4,5-trichlorophenoxyacetic acid (2,4,5-T), phenylacetic acid (PAA), kinetin (KIN), 6-benzylaminopurine (6BA), N6-(2-isopentenyl) adenine (2iP), zeatin (ZEA), gibberellin A1 (GA1), gibberellic acid (GA3), gibberellin A4 (GA4), gibberellin A7 (GA7), ethylene, brassinolide (BR), and jasmonic acid (JA).

In certain aspects, the callus growth medium can be a liquid at about 25° C. In some embodiments, the callus growth medium can be not a liquid at about 25° C. In some embodiments, the callus growth medium can be a semi-solid medium (such as gelled) at 25° C. The present Inventors discovered that a semi-solid medium provides better results than a liquid medium. Non-limiting examples of a semi-solid medium include soft agar, soft agarose, soft methylcellulose, xanthan gum, gellan gum, carrageenan, isabgol, guar gum, other soft polymeric gels, or any other gelling agent known in the art. In some embodiments, the callus growth medium can comprise agar. In some embodiments, the callus growth medium can be agar-free. In some embodiments, the callus growth medium is free of any gelling agent. In some embodiments, the callus growth medium that is agar- or gelling agent-free can be a liquid. In some embodiments, the callus growth medium that is agar- or gelling agent-free can be a solid. In some embodiments, the callus growth medium that is agar-free can be a gel. In some embodiments, the callus growth medium that is agar-free can comprise an agar-substitute.

In some embodiments, the elongation medium can have a pH. The pH of the elongation medium can be appropriate for producing/inducing an elongated cell, such as an elongated cotton cell or a plurality of elongated cotton cells. In some embodiments, the pH of the elongation medium can be optimized for cell elongation (such as cotton cell elongation). In some embodiments, the pH of the elongation medium can be from 5.3 to 6.3. In some embodiments, the pH of the elongation medium can be, or be about, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, or 6.9, or a range between any two foregoing values.

The present invention includes elongation mediums, and their use in the in vitro methods for producing cotton fiber. In some embodiments, the maturation mediums described herein can facilitate or promote maturation of cells, such as maturation of cotton cells. A maturation medium can comprise one or more salts, macronutrients, micronutrients, organic molecules, and/or hormones (such as those that can facilitate or promote maturation). In some embodiments, the maturation medium can comprise a maturation reagent. In some embodiments, the maturation reagent of the maturation medium can be a wall-regeneration reagent.

The present invention includes the use of proliferating cell aggregates, and their creation, for use in the in vitro methods of cotton fiber production. In some embodiments, the plant cell composition as described hereinbelow or described anywhere else herein can be derived from the proliferating cell aggregate. The proliferating cell aggregate can be an aggregate of plant cells that are proliferating. Proliferating cells in an aggregate can be attached or connected to each other, for example, via cell to cell interactions. The proliferating cell aggregate can be a friable callus is friable, which is not sticky or soft, but is also not so hard or dense that it cannot be physically broken or crumbled. A friable callus thus differs “a hard callus”, which is compact and brittle, and thus not amenable to being broken or crumbled. Preferably the callus is a friable callus. The present Inventors discovered that a friable callus may have individual cells dissociated from the callus using simple mechanical manipulation.

Proliferating cells can be of one type (a homogenous aggregate) or of two or more types (a heterogeneous aggregate). The proliferating cell aggregate can be a mixed aggregate (e.g., where cell types are mixed together), a clustering aggregate (e.g., where cells of different types are tending toward different parts of the aggregate), or a separating aggregate (where cells of different types are pulling apart from each other). Cells of the proliferating cell aggregate can divide at a rate greater than a cell division rate of remaining cells in said plant callus. In some embodiments, cells of the proliferating cell aggregate can divide at a rate that can be at least 2, 3, 4, 5, 6, 7, 8, 9, 10, 20, 30, 40, or 50 times greater than a cell division rate of plant callus cells.

The present invention includes the use of cells from a cotton plant cell callus and methods for preparing such a callus. The plant callus can be a growing mass of plant parenchyma cells. However, the Inventors discovered that, surprisingly, cells from any meristematic part of a cotton plant are sufficient for callus induction. Thus, the plant callus can be created using cells obtained or derived from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC). In some cases, the mass of plant parenchyma cells can be unorganized. The plant callus can be collected from cells covering the wound of a plant or plant part. Preferably, the plant callus is created by inducing a plant tissue sample (e.g., an explant) with a callus induction medium. In some cases, induction of an explant can occur after surface sterilization and plating onto a medium in vitro (e.g., in a closed culture vessel such as a Petri dish). Induction can comprise supplementing the medium with plant growth regulators, such as auxins, cytokinins, or gibberellins to initiate callus formation. Induction can be performed at a temperature of, or of about, 20° C., 25° C., 28° C., 30° C., 35° C., or 40° C., or a range between any two foregoing values.

Compositions comprising cotton plant cells are included in the present invention. The plant cell compositions described herein can be a final product of a method for preparation of cell bank stocks provided herein. The plant cell compositions can be compositions of engineered cells, or a compositions of wildtype cells. The plant cell compositions can be cell bank stocks. The plant cell compositions can comprise a plurality of plant cells obtained by growing a callus in a growth medium to produce a proliferating cell aggregate followed by culturing the proliferating cell aggregate.

The plant cell compositions described herein can be in a growth phase. The growth phase can comprise cell division, cell enlargement, and/or cell differentiation. The growth phase comprising cell division can be an exponential growth phase (e.g., dowaiting). In some embodiments, the exponential growth phase can occur as cells are mitotic. In some embodiments, during exponential growth, each generation of cells can be twice as numerous as the previous generation. In some embodiments, not all cells may survive in a given generation. In some embodiments, each generation of cells can be less than twice as numerous as the previous generation. In some embodiments, the exponential growth phase can be determined (e.g., quantified or identified) by a cell viability assay. In some embodiments, another aspect of the plant cell composition can be determined by a cell viability assay. In some embodiments, the cell viability assay can be an assay that can determine the ability of a cell to maintain or recover viability. In some embodiments, the cells of the plant cell composition can be assayed for their ability to divide or for active cell division. In some embodiments, the cell viability assay can be an ATP test, calcein AM, clonogenic assay, ethidium homodimer assay, Evans blue, fluorescein diacetate hydrolysis/propidium iodide staining (FDA/PI staining), flow cytometry, formazan-based assays (e.g., MTT or XTT), green fluorescent protein based assays, lactate dehydrogenase (LDH) based assays, methyl violet, neutral red uptake, propidium iodide, resazurin, trypan blue, or a TUNEL assay. In some embodiments, the cell viability assay can determine a cytoplasmic level of diphenol compounds in the plant cell composition.

In some embodiments, the cotton (or engineered cotton) described herein can be derived from a Gossypium species. The Gossypium species can be selected from the group consisting of G. arboreum, G. anomalum, G. armourianum, G. klotzchianum, and G. raimondii. The cotton (or engineered cotton) can be derived from a Gossypium species selected from the group consisting of G. hirsutum, G. arboreum, G. barbadense, G. anomalum, G. armourianum, G. klotzchianum, and G. raimondii. The cotton (or engineered cotton) can be Gossypium hirsutum, Gossypium barbadense, Gossypium arboretum, Gossypium herbaceum, or another species of cotton.

The cotton cells used in the methods for producing cotton fiber in vitro may include the use of cotton cells that have a differently expressed gene (DEG). Cotton cells of the present disclosure can be subject to a mutagenic process to give rise the DEG. This process can occur in vitro and without ever growing a whole cotton plant with the DEG.

Mutagenesis can be achieved by radiation and/or chemical means, including EMS or sodium azide treatment of seed, or gamma irradiation. Chemical mutagenesis favors nucleotide substitutions rather than deletions. Heavy ion beam (HIB) irradiation is a known technique for mutagenesis. Ion beam irradiation has two physical factors, the dose (gy) and LET (linear energy transfer, keV/um) for biological effects that determine the level of DNA damage and the size of any DNA deletion(s), and these can be adjusted according to change the extent of mutagenesis.

Biological agents can also be used to create site-specific mutations in cotton cells. These agents may include enzymes that cause double stranded breaks in DNA, which stimulate endogenous repair mechanisms. These enzymes include endonucleases, zinc finger nucleases, transposases and site-specific recombinases.

In certain aspects, the cotton cells may be transgenic. A transgenic cell is a genetically modified plant cell in which an endogenous genome or gene is supplemented or modified by an introduced foreign or exogenous gene or sequence. Often, these genes are under the control of a promoter to which they are operably connected. A transgene is a foreign exogenous gene or sequence introduced into the plant.

Also provided herein are bioreactors configured to produce any one or more compositions associated with the in vitro production of fiber as disclosed herein

In some embodiments, a bioreactor can be configured to produce a cell bank stock. In some embodiments, a bioreactor can be configured to carry out a method for preparing a cell bank stock. In some such cases, a bioreactor can be configured to utilize components of a kit for preparation of a cell bank stock, such as a callus growth medium and/or a multiplication medium.

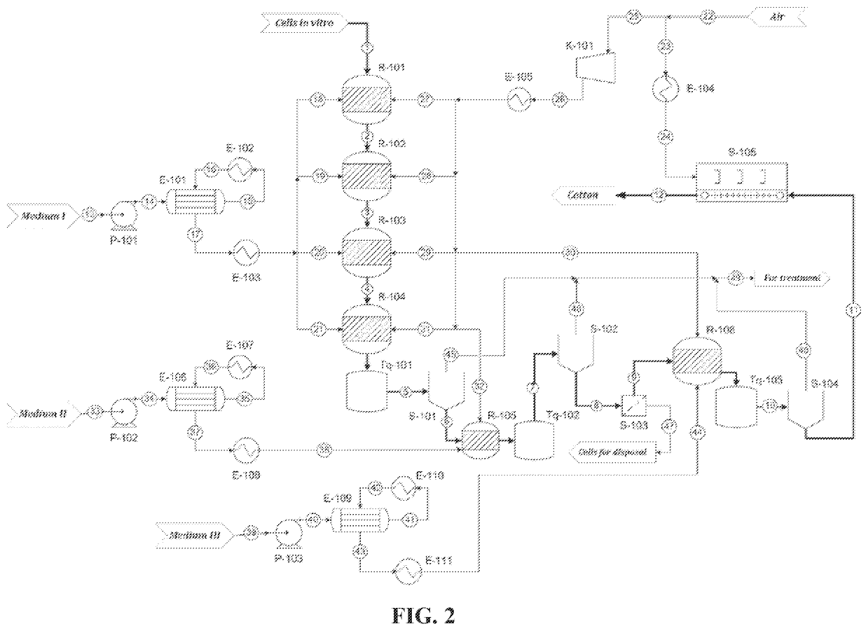

FIG. 2 provides a flow chart illustrating an example of different processes that can be performed by a bioreactor, and how these processes can be interconnected.

In some embodiments, a bioreactor can be configured to produce a cotton fiber. In some embodiments, a bioreactor can be configured to carry out a method for large scale cotton fiber production. In some embodiments, a bioreactor can be configured to carry out a method for rapid cotton fiber production. In some embodiments, a bioreactor can be configured to utilize components of a kit for large scale fiber production. In some embodiments, a bioreactor can be configured to utilize components of a kit for rapid fiber production.

In some embodiments, a bioreactor can be configured to produce engineered cotton. In some embodiments, a bioreactor can be configured to utilize components of a kit for production of engineered cotton, which can comprise elements of kits provided herein.

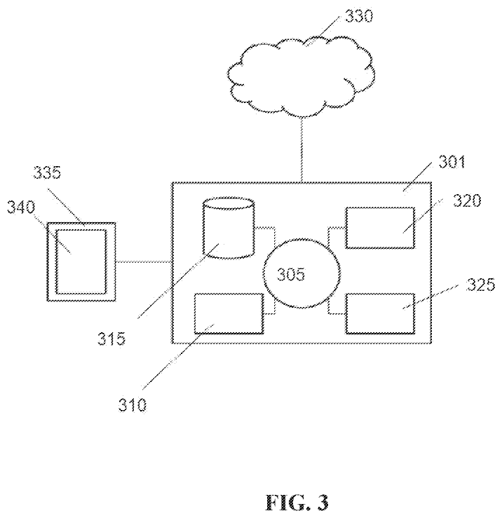

The present invention includes computer systems that are programmed to implement methods of the disclosure. FIG. 3 shows a computer system 301 that is programmed or otherwise configured to provide and/or implement instructions for or means of implementation of induction, callus growth, cell culture, elongation, or maturation. The computer system 301 can regulate various aspects of induction, callus growth, cell culture, elongation, or maturation of the present disclosure. The computer system 301 can be an electronic device of a user or a computer system that is remotely located with respect to the electronic device. The electronic device can be a mobile electronic device.

The computer system 301 includes a central processing unit (CPU, also “processor” and “computer processor” herein) 305, which can be a single core or multi core processor, or a plurality of processors for parallel processing. The computer system 301 also includes memory or memory location 310 (e.g., random-access memory, read-only memory, flash memory), electronic storage unit 315 (e.g., hard disk), communication interface 320 (e.g., network adapter) for communicating with one or more other systems, and peripheral devices 325, such as cache, other memory, data storage and/or electronic display adapters. The memory 310, storage unit 315, interface 320 and peripheral devices 325 are in communication with the CPU 305 through a communication bus (solid lines), such as a motherboard. The storage unit 315 can be a data storage unit (or data repository) for storing data. The computer system 301 can be operatively coupled to a computer network (“network”) 330 with the aid of the communication interface 320. The network 330 can be the Internet, an internet and/or extranet, or an intranet and/or extranet that is in communication with the Internet. The network 330 in some cases is a telecommunication and/or data network. The network 330 can include one or more computer servers, which can enable distributed computing, such as cloud computing. The network 330, in some cases with the aid of the computer system 301, can implement a peer-to-peer network, which may enable devices coupled to the computer system 301 to behave as a client or a server.

The CPU 305 can execute a sequence of machine-readable instructions, which can be embodied in a program or software. The instructions may be stored in a memory location, such as the memory 310. The instructions can be directed to the CPU 305, which can subsequently program or otherwise configure the CPU 305 to implement methods of the present disclosure. Examples of operations performed by the CPU 305 can include fetch, decode, execute, and writeback.

The CPU 305 can be part of a circuit, such as an integrated circuit. One or more other components of the system 201 can be included in the circuit. In some cases, the circuit is an application specific integrated circuit (ASIC).

The storage unit 315 can store files, such as drivers, libraries and saved programs. The storage unit 315 can store user data, e.g., user preferences and user programs. The computer system 301 in some cases can include one or more additional data storage units that are external to the computer system 301, such as located on a remote server that is in communication with the computer system 301 through an intranet or the Internet.

The computer system 301 can communicate with one or more remote computer systems through the network 330. For instance, the computer system 301 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 301 via the network 330.

Methods as described herein can be implemented by way of machine (e.g., computer processor) executable code stored on an electronic storage location of the computer system 301, such as, for example, on the memory 310 or electronic storage unit 315. The machine executable or machine-readable code can be provided in the form of software. During use, the code can be executed by the processor 305. In some cases, the code can be retrieved from the storage unit 315 and stored on the memory 310 for ready access by the processor 305. In some situations, the electronic storage unit 315 can be precluded, and machine-executable instructions are stored on memory 310.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 301, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 301 can include or be in communication with an electronic display 335 that comprises a user interface (UI) 340 for providing, for example, instructions for or means of implementation of induction, callus growth, cell culture, elongation, or maturation. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 305. The algorithm can, for example, provide and/or execute instructions for or means of implementation of induction, callus growth, cell culture, elongation, or maturation.

The present invention is further described by the following non-limiting Examples.

### EXAMPLES

### Example 1: Preparation of a Plant Cell Composition

From a select plant (e.g., cotton), cells are isolated by placing sterilized explants from apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and cambial meristematic cells (CMC) on a callus induction medium (e.g., a semi-solid basal salts medium) for induction. The dedifferentiated masses formed are conditioned by passing three up to five subculturing at intervals of 21-26 days on a callus growth medium (e.g., a semi-solid basal salts medium) for growth.

After cell culture stabilization, cells from a soft or friable callus are transferred into a liquid medium to form a suspension cell. Suspensions are subcultured at intervals of 15-20 days for homogenization to provide fine cell suspension culture, by filtering, pipetting/decantation, or by addition of a low concentration of pectinase. The homogeneous nature of cells in these cultures give rise to reproducible and reliable results.

### Example 2: Cryopreservation of Suspension-Cultured Cells

Cryopreservation techniques remove the need for frequent culturing and, thus, reduce the chance of microbial contamination. The protocol provided below allows the cryopreservation of over 100 cell lines simultaneously in a single day.

Suspension-cultured cells from Gossypium spp. and other species in exponentially growing phase are transferred to 15 ml tubes and centrifuged at 100×g for 1 min. Cell suspensions are handled using micropipettes with large orifice tips. The supernatant is removed, and cells are then suspended in cryoprotectant solution (LS: 2M glycerol, 0.4M sucrose) supplemented with up to 100 mM L-proline at the cell density of 10% (v/v), and incubated at room temperature for 0-120 minutes with and without shaking at 60 rpm. Aliquots (0.5 ml) of cell suspensions are dispensed into cryovials (Fisher Scientific). Cryovials containing cell suspension in LS are cooled to −35° C. at a rate of −0.5, −1, or −2° C. min⁻¹ using a programmable freezer. After reaching −35° C., cells are kept at −35° C. for 0, 30, or 60 minutes, and then plunged into liquid nitrogen.

In vitro dedifferentiated plant cell suspension cultures are more convenient for large-scale production, as they offer the advantage of a simplified model system for the study of plants. Cell suspension cultures contain a relatively homogeneous cell population, allowing rapid and uniform access to nutrition, precursors, growth hormones, and signal compounds for the cells.

### Example 3: Cell Recovery

The vials containing cryopreserved cells are transferred from the liquid nitrogen storage vessel into a Dewar flask containing liquid nitrogen. Each vial is transferred (one by one) to a clean 35-40° C. water bath and gently flipped several times until thawed (the last piece of ice disappears). Immediately, each vial is placed on ice again. Each vial is centrifuged at 100 g, at 4° C. for 1-2 min. The outside of each vial is wiped with 70% (vol/vol) ethanol and the supernatant from each vial is removed using a sterile Pasteur pipette. A sterile 3.5-ml transfer pipette is used to transfer two-thirds' volume of the cells by spreading or placing them as a few clusters onto the filter paper. The dish is closed and sealed with Parafilm.

The dish(es) are covered with one or two sheets of filter paper to reduce the light intensity then placed in the culture room in regular conditions (24-26° C.). After 2 days of recovery, a spatula (width of 4 mm) is used to collect some cell mass (about 100-200 mg FW) from the plate and place into a microtube for viability testing. The remaining cells are transferred with the upper filter paper to a fresh recovery dish containing recovery medium. The dishes are closed and sealed, covered with filter paper, and then returned to the culture room.

Depending on their growth rates, cells are allowed to grow for an additional number of days in the same culture room, in regular conditions (24-26° C.). When most of the filter paper is covered with a thick layer of cells, the cell mass is transferred to a fresh dish containing recovery medium without filter paper for a further 1-2 weeks under standard conditions (at this recovery stage, agarose may be replaced by agar or another gelling agent). After a recovery period of 3-9 weeks, cells are transferred to a liquid medium to initiate suspension culture.

### Example 4: Bioreactor Inoculation

For inoculum, the medium is prepared with deionized (DI) water to make a total volume of 200 mL (1 L flask) and sterilized through autoclaving at 121° C. for 15 minutes. After cooling to room temperature, plant growth regulators and amino acids are added using a 0.2 μm pore size membrane filter. Twenty grams of cells are inoculated and maintained in a shaker in dark at a temperature from about 30° C. to about 35° C. at 80 rpm and left for inoculum growth. After 16 days (7 days of LAG phase and 9 days of exponential phase), the suspension is sufficiently dense for feeding the bioreactors (Titer=100 g L⁻¹, comparable a thick applesauce with no visible free medium).

An illustrative schematic of the bioreactor can be found in FIG. 2. The bioreactor is fed with in vitro cells, with sterilized medium, and air compression. The bioreactors are connected to the controller prior to inoculation, to stabilize pH 5.8 (±0.2) and to control and calibrate the flow of O₂. As illustrated in the flowchart in FIG. 2, the first vessel of the inoculum train occurs at a temperature from about 30° C. to about 35° C. with a 100 g L⁻¹ of cells at an exponential phase. In parallel, the sterilization of the culture medium occurs at approximately 125 to approximately 140° C. and returns (stream 16) to the heat exchanger (stream 13) to cooling the medium at a temperature from about 30° C. to about 35° C. (E-103). With this, the sterile medium is ready to feed the reactors of the multiplication area (reactors R-101 to R-104).

The air for cell oxygenation is also adjusted to the process temperature in the heat exchanger (E-105) and thus is split into four different streams (streams 27, 28, 29 and 31) that feed the inoculum train (reactors R-101 to R-104).

The multiplication occurs in a duration from 5 to 12 days for cells, and the duplication time is approximately 1 day to 3 days (depending on linage(s)). These times conclude when the cell amount increases, for example, 64 times. In the end, the content is loaded to the next reactor (R-102) and so on. The last reactor (R-104) has an adjacent lung tank, where after the reaction the contents are discharged in the batch feeding tank (Tq-101) with continuous output (stream 5). Thus, during the multiplication time of the R-104 reactor, the Tq-101 is continuously unloading the cells for the next stage, the separation, at a continuous flow rate.

Table B, below, provides experimental results showing the success of inoculating a bioreactor using cotton cells in accordance with the methods disclosed herein. Table B provides details regarding the cotton varieties from which the cells were obtained, the cell growth medium which was inoculated in the bioreactor, and other relevant conditions.

TABLE B

| | | | Testing | Growth | Preliminary |
|---|---|---|---|---|---|
| Variety Name | Quantity | Volume | medium | Conditions | Growth Result |
| PD 2164 | 40 mg | 8 ml in a | 1xMS, 1xB5, | 30° C., dark, | poor |
| | | 25 ml bottle | 30 g/L glucose, | 180 RPM | |
| PD 2164 | 37 mg | 30 ml in a | 0.548 mg/L | 30° C., dark, | poor |
| | | 125 ml flask | Kinetin, | 180 RPM | |
| | | | 0.1 mg/L 2,4-D | | |
| Acala MAXXA | 53 mg | 8 ml | 1xMS, 1xB5, | 30° C., dark, | poor |
| | | | 30 g/L glucose, | 180 RPM | |
| Acala MAXXA | 31 mg | 30 ml | 0.548 mg/L | 30° C., dark, | poor |
| | | | Kinetin, | 180 RPM | |
| | | | 0.1 mg/L 2,4-D | | |
| FJA | 48 mg | 8 ml | 1xMS, 1xB5, | 30° C., dark, | poor |
| | | | 30 g/L glucose, | 180 RPM | |
| FJA | 41 mg | 30 ml | 0.548 mg/L | 30° C., dark, | poor |
| | | | Kinetin, | 180 RPM | |
| | | | 0.1 mg/L 2,4-D | | |
| Pima S-7 | 34 mg | 8 ml | 1xMS, 1xB5, | 30° C., dark, | poor |
| | | | 30 g/L glucose, | 180 RPM | |
| Pima S-7 | 48 mg | 30 ml | 0.548 mg/L | 30° C., dark, | poor |
| | | | Kinetin, | 180 RPM | |
| | | | 0.1 mg/L 2,4-D | | |
| Pima S-7 | 91 mg | 30 ml | 1xMS, 1xB5, | 30° C., dark, | poor |
| | | | 30 g/L glucose, | 180 RPM | |
| | | | 0.548 mg/L | | |
| | | | Kinetin | | |
| Pima S-7 | 110 mg | 30 ml | 1xMS, 1xB5, | 30° C., dark, | good |
| | | | 30 g/L glucose, | 180 RPM | |
| | | | 0.1 mg/L 2,4-D | | |
| Pima S-7 | 82 mg | 30 ml | 1xMS, 1xB5, | 30° C., light, | poor |
| | | | 30 g/L glucose, | 180 RPM | |
| | | | 0.548 mg/L | | |
| | | | Kinetin, | | |
| | | | 0.1 mg/L 2,4-D | | |
| Pima S-7 | | 8 ml in a | 1xMS, 1xB5, | 30° C., dark, | good |
| | | 50 ml | 30 g/L Sucrose | 180 RPM | |
| | | flask | 0.1 mg/L | | |
| | | | Kinetin, 1 mg/L | | |
| | | | 2,4-D, 100ml/L | | |
| | | | coconut | | |
| | | | water(B105) | | |
| Pima S-7 | | 8 ml in a | 1xMS, 1xB5, | 30° C., dark, | good |
| | | 50 ml | 20 g/L Sucrose, | 180 RPM | |
| | | flask | 0.1 mg/L | | |
| | | | Kinetin, 1 mg/L | | |
| | | | 2,4-D, 100 ml/L | | |
| | | | coconut | | |
| | | | water(B50) | | |
| Pima S-7 | | 8 ml in a | 1xMS, 1xMS, | 30° C., dark, | good |
| | | 50 ml | 30 g/L Sucrose, | 180 RPM | |
| | | flask | 0.18 mg/L | | |
| | | | NAA, 0.2 mg/L | | |
| | | | 2,4-D(M42) | | |

As shown in Table B, inoculating the bioreactor is far more efficient when done under dark conditions as opposed to light. Accordingly, the present disclosure provides methods of inoculating a bioreactor using a cotton cell culture, wherein the inoculation occurs under dark conditions.

As shown in Table B, the composition of the growth medium used when inoculating a bioreactor has an impact on cell growth. Thus, the present disclosure provides methods of inoculating a bioreactor with cotton cells, wherein the growth medium comprises plant hormones or growth regulators. As shown in Table B, when the growth medium included 2,4-dichlorophenoxyacetic acid (2,4-D), growth improved. Thus, the present disclosure provides methods of inoculating a bioreactor with cotton cells, wherein the growth medium comprises 2,4-dichlorophenoxyacetic acid (2,4-D).

As shown in Table B, the methods of the present disclosure allowed successful cell growth when inoculating a bioreactor with cells from all cotton varieties tested. Thus, the present disclosure provides methods of inoculating a bioreactor with any of the cotton varieties disclosed herein. In certain embodiments, the cotton cells used to inoculate a bioreactor in accordance with the methods disclosed herein are derived and/or obtained, in whole or in part, from at least one cotton plant of a variety selected from PAYMASTER HS26, PD 2164, SA 2413, SEALAND #1 (G.B. X G.H.), SOUTHLAND M1, STATION MILLER, TASHKENT 1, TIDEWATER 29 (G.B. X G.H.), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, or a progeny of any thereof. In certain embodiments, the cotton cells used to inoculate a bioreactor in accordance with the methods disclosed herein are derived and/or obtained, in whole or in part, from at least one cotton plant of a variety selected from PD 2164, Acala MAXXA, FJA, Pima S-7, or a progeny of any thereof.

As shown in Table B, surprisingly, cells from cotton variety Pima S-7 provided good growth when inoculating a bioreactor. This included when using milligram quantities of cotton cells to form an inoculum and across a range of growth mediums. Unexpectedly, Pima S-7 provided superior growth/inoculation compared to Acala MAXXA and FJA. Moreover, this superior growth occurred even when using the same growth medium. For example, as shown in Table B, Pima S-7 provided good growth, while Acala MAXXA and FJA showed poor growth when all were cultured using a growth medium with the same concentrations MS, B5, glucose, Kinetin, and 2,4-D. Accordingly, the present disclosure provides inoculating a bioreactor with cells derived and/or obtained, in whole or in part, from a cotton plant of the Pima S-7 variety, or a progeny thereof.

### Example 5: Elongation of Cells

For elongation, plant cells are separated from the medium using a decanter vessel (S-101) (stream 6) and the medium can be relocated for water treatment (stream 45), as illustrated in the flowchart in FIG. 2. The elongation growth medium is added to the reactors to sterilization by autoclaving at same conditions used in multiplication step and cooling at a temperature from about 30° C. to about 35° C. for cell differentiation.

Thus, the cells from the multiplication (stream 6) feed three elongation reactors (R-105, R-106, and R-107) are represented by the reactor block (R-105) in the flowchart in FIG. 2. Each reactor receives a third of the cells and the reaction volume comprises the cells (stream 6), medium (stream 38), and air (stream 32) flows.

### Example 6: Separation and Isolation of Elongated Cells

After elongation according to Example 5, 3 tanks (Tq-102, Tq-103, and Tq-104) are fed, which in the flowchart in FIG. 2 are represented only by block Tq-102. Each tank, with volume slightly larger than those of the reactors, receives the substantially same volume of the three reactors. The output of the elongation tanks (stream 7) is routed to the second decanter (S-102). The bottom product (stream 8), comprising elongated and unelongated cells, is routed to the sieve (S-103), while the medium (stream 46) is removed to the effluent treatment. The function of the sieve is to remove unelongated and smaller cells that are not pre-fibers. The sieve (S-103) retains the elongated cells (pre-fibers) and releases all nonelongated cells (which will not become cotton fibers).

### Example 7: Maturation and Drying of Cells

In the maturation stage, as well as in the multiplication and elongation stages, a sterilized medium is used. Maturation is recognized by secondary cell wall deposition. Sugars are combined to produce cellulose, which is the main component of cotton fiber (natural glucose polymerization) that occurs inside the cell forming the secondary wall. In this process, the density of pre-fiber increases from 1.05 to 1.55 g/ml, which is the density of cotton fiber.

After maturation time, the R-108 output is directed to the buffer tank Tq-105 (FIG. 2) to enable a continuous downstream process. In the sequence, the mid-fiber mixture (stream 10) is routed to the third decanter (S-104), where the cotton fibers (stream 11) are separated from the medium (stream 48). At this stage, the fibers produced have moisture content above acceptable level (10 to 20% in water mass). To reduce the moisture content, a drying process working with air is implemented. This air passes through the cotton fibers and part of the water is removed until a moisture content of at most 5% is reached.

### Example 8: Recycling

In some embodiments, a composition created via a method described herein can be recycled. For example, in such a case, after completion of a method or step of a method, an aliquot of a composition is reserved and re-introduced into an earlier step in a method. In some cases, an aliquot of cells unsuccessful in induction, growth, elongation, or maturation is reserved and re-introduced into an earlier step in a method.

### Example 9: Production of Cotton Fiber from Cotton Ovule Cells

Tables C, D, and E, below, show the results of growth and elongation of cotton cell cultures in accordance with the methods disclosed herein. For each growth result, cotton ovule cells, which may include ovule epidermal cells, were mechanically extracted from a cotton boll.

The extracted cotton ovule cells were cultured, multiplied, and in some cases, elongated. Each table provides the genotype/cultivar and variety name from which the cotton cell cultures were originally obtained. The tables also provide the ovule location from which the cells were taken from a parental cotton plant for the cell cultures.

TABLE C
Cotton cell culture results from cells
cultured from an upper ovule location.

| | Ovule | |
|---|---|---|
| Variety Name | location | Growth Results |
| PAYMASTER HS26 | Upper | some |
| PD 2164 | Upper | excellent |
| SA 2413 | Upper | poor |
| SEALAND #1 (G.B. × G.H.) | Upper | some/fiber |
| SOUTHLAND M1 | Upper | excellent |
| STATION MILLER | Upper | some |
| TASHKENT 1 | Upper | excellent |
| TIDEWATER 29 (G.B. × G.H.) | Upper | excellent |
| TOOLE | Upper | poor |
| WESTERN STORMPROOF | Upper | some |
| ACALA 5 | Upper | excellent/fiber |
| ALLEN 33 | Upper | some |
| CD3HCABCUH-1-89 | Upper | good |
| DELTAPINE 14 | Upper | some |
| DES 24 | Upper | some |
| DES 56 | Upper | poor |
| DIXIE KING | Upper | excellent |
| FJA | Upper | excellent |
| M.U.8B UA 7-44 | Upper | some |
| NC 88-95 | Upper | some |
| PAYMASTER HS200 | Upper | excellent |
| Pima S-7 | Upper | some |
| Acala MAXXA | Upper | excellent |
| Coasland 320 | Upper | poor |

TABLE D
Cotton cell culture results from cells
cultured from a middle ovule location.

| | Ovule | |
|---|---|---|
| Variety Name | location | Growth |
| PAYMASTER HS26 | Middle | some |
| PD 2164 | Middle | excellent |
| SA 2413 | Middle | some/fiber |
| SEALAND #1 (G.B. × G.H.) | Middle | some/fiber |
| SOUTHLAND M1 | Middle | excellent |
| STATION MILLER | Middle | some |
| TASHKENT 1 | Middle | excellent |
| TIDEWATER 29 (G.B. × G.H.) | Middle | excellent |
| TOOLE | Middle | some/fiber |
| WESTERN STORMPROOF | Middle | some |
| ACALA 5 | Middle | some |
| ALLEN 33 | Middle | some |
| CD3HCABCUH-1-89 | Middle | good |
| DELTAPINE 14 | Middle | some |
| DES 24 | Middle | some |
| DES 56 | Middle | poor |
| DIXIE KING | Middle | excellent |
| FJA | Middle | excellent |
| M.U.8B UA 7-44 | Middle | some/fiber |
| NC 88-95 | Middle | some |
| PAYMASTER HS200 | Middle | excellent |
| Pima S-7 | Middle | some |
| Acala MAXXA | Middle | excellent |
| Coasland 320 | Middle | poor |

TABLE E
Cotton cell culture results from cells
cultured from a bottom ovule location.

| | Ovule | |
|---|---|---|
| Variety Name | location | Growth |
| PAYMASTER HS26 | Bottom | some |
| PD 2164 | Bottom | excellent |
| SA 2413 | Bottom | some |
| SEALAND #1 (G.B. × G.H.) | Bottom | poor |
| SOUTHLAND M1 | Bottom | excellent |
| STATION MILLER | Bottom | some |
| TASHKENT 1 | Bottom | excellent |
| TIDEWATER 29 (G.B. × G.H.) | Bottom | excellent |
| TOOLE | Bottom | some/fiber |
| WESTERN STORMPROOF | Bottom | good |
| ACALA 5 | Bottom | excellent/fiber |
| ALLEN 33 | Bottom | some |
| CD3HCABCUH-1-89 | Bottom | excellent |
| DELTAPINE 14 | Bottom | some |
| DES 24 | Bottom | some |
| DES 56 | Bottom | poor |
| DIXIE KING | Bottom | excellent/fiber |
| FJA | Bottom | excellent |
| M.U.8B UA 7-44 | Bottom | some/fiber |
| NC 88-95 | Bottom | some |
| PAYMASTER HS200 | Bottom | excellent |
| Pima S-7 | Bottom | excellent |
| Acala MAXXA | Bottom | excellent |
| Coasland 320 | Bottom | poor |

As shown in Tables C, D, E, all varieties were successfully grown in accordance with the methods of the present disclosure. Thus, the present disclosure provides cotton, methods of growing cotton in accordance with any of the method/protocols provided herein, and persistent cell lines, wherein the cotton cells are derived and/or obtained, in whole or in part, which can be used across a range of cotton species and varietals. Accordingly, the in vitro methods of cotton production can use cotton cells derived from a cotton plant of any varietal, including one selected from PAYMASTER HS26, PD 2164, SA 2413, SEALAND #1 (G.B. X G.H.), SOUTHLAND M1, STATION MILLER, TASHKENT 1, TIDEWATER 29 (G.B. X G.H.), TOOLE, WESTERN STORMPROOF, ACALA 5, ALLEN 33, CD3HCABCUH-1-89, DELTAPINE 14, DES 24, DES 56, DIXIE KING, FJA, M.U.8B UA 7-44, NC 88-95, PAYMASTER HS200, Pima S-7, Acala MAXXA, Coasland 320, and or a progeny of any thereof.

As shown in Tables C, D, and E, certain varieties produced good or excellent growth. Thus, in certain embodiments, the cotton cells are derived and/or obtained, in whole or in part, from at least one cotton plant of a variety selected from PD 2164, SOUTHLAND M1, ACALA 5, CD3HCABCUH-1-89, FJA, TASHKENT 1, WESTERN STORMPROOF, PAYMASTER HS200, Pima S-7, TIDEWATER 29, DIXIE KING, FJA, SOUTHLAND M1, and Acala MAXXA, or a progeny of any thereof. Certain varieties produced excellent growth. Thus, in certain embodiments, the cotton cells are derived and/or obtained, in whole or in part, from at least one cotton plant of a variety selected from PD 2164, ACALA 5, SOUTHLAND M1, CD3HCABCUH-1-89, FJA, Pima S-7, and Acala MAXXA, or a progeny of any thereof.

As shown in Table C, certain varieties produced good or excellent growth using cells obtained from an ovule, which may include ovule epidermal cells, located on the upper/top third of a boll, e.g., distal from the location on the boll to which it connects or connected to the stem of a cotton plant. Thus, in certain embodiments, the cotton cells are derived and/or obtained, in whole or in part, from ovule cells and/or ovule epidermal cells obtained from the top third of a cotton boll from at least one cotton plant of a variety selected from PD 2164, SOUTHLAND M1, ACALA 5, Acala MAXXA, FJA, TASHKENT 1, PAYMASTER HS200, TIDEWATER 29, DIXIE KING, and CD3HCABCUH-1-89, or a progeny of any thereof. As shown in Table C, certain varieties produced excellent growth using ovule cells and/or ovule epidermal cells obtained from the top third of a cotton boll. Thus, in certain embodiments, the cotton cells are derived and/or obtained, in whole or in part, from ovule and/or ovule epidermal cells obtained from the top third of a cotton boll from at least on cotton plant of a variety selected from PD 2164, SOUTHLAND M1, ACALA 5, Acala MAXXA, FJA, TASHKENT 1, PAYMASTER HS200, TIDEWATER 29, and DIXIE KING, or a progeny of any thereof.

As shown in Table D, certain varieties produced good or excellent growth using ovule cells and/or ovule epidermal cells obtained from the middle third of the cotton boll. Thus, in certain embodiments, the cotton cells are derived and/or obtained, in whole or in part, from ovule cells and/or ovule epidermal cells obtained from the middle third of a cotton boll from a cotton plant of a variety selected from at least one cotton plant of a variety selected from PD 2164, SOUTHLAND M1, CD3HCABCUH-1-89, Acala MAXXA, FJA, TASHKENT 1, PAYMASTER HS200, TIDEWATER 29, and DIXIE KING, or a progeny of any thereof.

As shown in Table D, certain varieties produced excellent growth using ovule cells and/or ovule epidermal cells obtained from the middle third of a cotton boll. Thus, in certain embodiments, the cotton cells are derived and/or obtained, in whole or in part, from ovule cells and/or ovule epidermal cells obtained from the middle third of a cotton boll from at least on cotton plant of a variety selected from PD 2164, SOUTHLAND M1, Acala MAXXA, FJA, TASHKENT 1, PAYMASTER HS200, TIDEWATER 29, and DIXIE KING, or a progeny of any thereof.

As shown in Table E, certain varieties produced good or excellent growth using ovule cells and/or ovule epidermal cells obtained from the bottom third of the boll. Thus, in certain embodiments, the cotton cells are derived and/or obtained, in whole or in part, from ovule cells and/or ovule epidermal cells obtained from the bottom third of a cotton boll from a cotton plant of a variety selected from at least one cotton plant of a variety selected from PD 2164, SOUTHLAND M1, TASHKENT 1, WESTERN STORMPROOF, ACALA 5, CD3HCABCUH-1-89, FJA, Pima S-7, Acala MAXXA, PAYMASTER HS200, TIDEWATER 29, and DIXIE KING, or a progeny of any thereof. As shown in Table E, certain varieties produced excellent growth using ovule cells and/or ovule epidermal cells obtained from the bottom third of a cotton boll. Thus, in certain embodiments, the cotton cells are derived and/or obtained, in whole or in part, from ovule cells and/or ovule epidermal cells obtained from the bottom third of a cotton boll from at least on cotton plant of a variety selected from PD 2164, SOUTHLAND M1, ACALA 5, TASHKENT 1, PAYMASTER HS200, TIDEWATER 29, DIXIE KING, CD3HCABCUH-1-89, FJA, Pima S-7, and Acala MAXXA, or a progeny of any thereof.

As shown in Tables C, D, and E, certain varieties produced good or excellent growth using cells from more than one ovule location. Thus, in certain embodiments, the cotton cells are derived and/or obtained, in whole or in part, from ovule cells and/or ovule epidermal cells obtained from at least one cotton plant of a variety selected from PD 2164, SOUTHLAND M1, ACALA 5, FJA, Acala MAXXA, DIXIE KING, TIDEWATER 29, PAYMASTER HS200, and TASHKENT 1, or a progeny of any thereof.

As shown in Tables C, D, and E, certain varieties quickly produced detectable levels of fiber in the grown cells. Accordingly, in certain embodiments, the cotton cells are derived and/or obtained, in whole or in part, from at least one cotton plant of a variety selected from SEALAND #1 (G.B. X G.H.), ACALA 5, SA 2413, TOOLE, M.U.8B UA 7-44, DIXIE KING, or a progeny of any thereof. As shown in Tables C, D, and E, certain varieties quickly produced detectable levels of fiber in the grown cells. As shown in Tables C and E, ACALA 5 showed both quickly detectable levels of fiber and excellent growth.

Table F, below, shows the results of growth and elongation of cotton cell cultures derived from specific varietals in accordance with the methods disclosed herein. For each growth result, cotton ovule cells, which may include ovule epidermal cells, are mechanically extracted from a cotton boll. The extracted cotton ovule cells are cultured, multiplied, and in some cases, elongated. Each table provides the genotype/cultivar and variety name from which the cotton cell cultures are originally obtained.

TABLE F
Cotton cell culture results

| Variety Name | Growth Results | Fiber |
|---|---|---|
| PD 2164 | excellent | yes |
| SOUTHLAND M1 | excellent | yes |
| TASHKENT 1 | excellent | yes |
| TIDEWATER 29 | excellent | yes |
| DIXIE KING | excellent | yes |
| FJA | excellent | yes |
| PAYMASTER HS200 | excellent | yes |
| Acala MAXXA | excellent | yes |

As shown in Table F, the selected varietals produce excellent growth using cells from ovules. Thus, in certain embodiments, the cotton cells are derived and/or obtained, in whole or in part, from ovule cells and/or ovule epidermal cells obtained from at least one cotton plant of a variety selected from PD2164, SOUTHLAND M1, FJA, PAYMASTER HS200, TIDEWATER 29, TASHKENT 1, DIXIE KING, and Acala MAXXA, or a progeny of any thereof.

As shown in Table F, cells from these selected varietals quickly produce detectable levels of fiber in the grown cells. Accordingly, in certain embodiments, the cotton cells are derived and/or obtained, in whole or in part, from at least one cotton plant of a variety selected from PD2164, SOUTHLAND M1, FJA, PAYMASTER HS200, TIDEWATER 29, TASHKENT 1, DIXIE KING, and Acala MAXXA, or a progeny of any thereof.

What is claimed is:

1. A method for producing cotton fiber, the method comprising:
inoculating a bioreactor with cotton cells;
multiplying the cells in the bioreactor;
elongating the multiplied cells; and
harvesting cotton fiber from the elongated cells,
wherein the cotton cells are derived and/or obtained from cotton plants of a varietal selected from PAYMASTER HS26, PAYMASTER HS200, PD 2164, SA 2413, SEALAND #1 (G.B. X G.H.), SOUTHLAND M1, STATION MILLER, TASHKENT 1, TIDEWATER 29 (G.B. X G.H.), TOOLE, WESTERN STORMPROOF, ACALA 5, ALLEN 33, CD3HCABCUH-1-89, DELTAPINE 14, DES 24, DIXIE KING, FJA, M.U.8B UA 7-44, NC 88-95, PAYMASTER HS200, Pima S-7, Acala and MAXXA, or a progeny of any thereof.

2. The method of claim 1, wherein the varietal is selected from PD2164, WESTERN STORMPROOF, CD3HCABCUH-1-89, TASHKENT 1, SOUTHLAND M1, ACALA 5, FJA, PAYMASTER HS200, Pima S-7, and Acala MAXXA, or a progeny of any thereof.

3. The method of claim 2, wherein the varietal is selected from PD 2164, SOUTHLAND M1, ACALA 5, CD3HCABCUH-1-89, FJA, Pima S-7, and Acala MAXXA, or a progeny of any thereof.

4. The method of claim 3, wherein the varietal is selected from PD 2164, SOUTHLAND M1, and CD3HCABCUH-1-89, or a progeny of any thereof.

5. The method of claim 4, wherein the varietal is PD 2164 or a progeny thereof.

6. The method of claim 1, wherein the bioreactor is inoculated with cotton ovule cells and/or ovule epidermal cells.

7. The method of claim 6, wherein the cotton ovule cells and/or ovule epidermal cells are obtained from a cotton boll.

8. The method of claim 6, wherein the cotton ovule cells and/or ovule epidermal cells are obtained from a bottom third, a middle third, or a top third of the cotton boll, wherein the bottom is a location on the boll to which its growth from a cotton plant stem began.

9. The method of claim 8, wherein the cotton ovule cells and/or ovule epidermal cells are obtained from the top third of the cotton boll, and the varietal is selected from PD 2164 and ACALA 5, or a progeny of any thereof.

10. The method of claim 8, wherein the cotton ovule cells and/or ovule epidermal cells are obtained from the middle third of the cotton boll, and the varietal is selected from PD 2164 and FJA, or a progeny of any thereof.

11. The method of claim 8, wherein the cotton ovule cells and/or ovule epidermal cells are obtained from the bottom third of the cotton boll, and the varietal is selected from PD 2164, SOUTHLAND M1, ACALA 5, CD3HCABCUH-1-89, FJA, Pima S-7, and Acala MAXXA, or a progeny of any thereof.

12. The method of claim 1, wherein the bioreactor is inoculated with cells from a proliferating cell aggregate.

13. The method of claim 12, wherein the varietal is Pima S-7 or a progeny thereof.

14. The method of claim 1, wherein the proliferating cell aggregate is a friable callus.

15. The method of claim 14, further comprising:
obtaining cells from a cotton explant; and
contacting the cells from the cotton explant with a callus induction medium to produce the friable callus.

16. The method of claim 15, wherein the cells from a cotton explant are from cotton apical meristems, cotyledons, young leaves, hypocotyls, ovules, ovule epidermal cells, stems, mature leaves, flower, flower stalks, floral whorls, roots, bulbs, germinated seeds, somatic and zygotic embryo, and/or cambial meristematic cells (CMC).

17. The method of claim 15, wherein the method further comprises:
dissociating cells from the friable callus;
culturing the dissociated cells; and
inoculating the bioreactor with the cultured dissociated cells.

18. The method of claim 17, wherein the method further comprises:
culturing the dissociated cells in a liquid or semi-solid medium to form a cell suspension;
cryopreserving the cell suspension; and
inoculating the bioreactor with the cryopreserved cell suspension.

19. The method of claim 18, wherein the method further comprises homogenizing the cell suspension to form a fine cell suspension.

20. The method of claim 1, wherein the method produces at least 1 kilogram of cotton fiber for every 4,000 liters of water used in the method.

21. An in vitro method for producing cotton fibers using cotton plant cells obtained and/or derived from cells of a cotton plant of a varietal selected from PD2164, WESTERN STORMPROOF, CD3HCABCUH-1-89, TASHKENT 1, SOUTHLAND M1, ACALA 5, FJA, PAYMASTER HS200, Pima S-7, and Acala MAXXA, or a progeny of any thereof.

22. The method of claim 1, wherein the varietal is selected from PD2164, SOUTHLAND M1, FJA, PAYMASTER HS200, TIDEWATER 29, TASHKENT 1, DIXIE KING, and Acala MAXXA, or a progeny of any thereof.

23. The method of claim 8, wherein the cotton ovule cells and/or ovule epidermal cells are obtained from the top third of the cotton boll, and the varietal is selected from PD2164, SOUTHLAND M1, FJA, PAYMASTER HS200, TIDEWATER 29, TASHKENT 1, DIXIE KING, and Acala MAXXA, or a progeny of any thereof.

24. An in vitro method for producing cotton fibers using cotton plant cells obtained and/or derived from cells of a cotton plant of a varietal selected from PD2164, SOUTHLAND M1, FJA, PAYMASTER HS200, TIDEWATER 29, TASHKENT 1, DIXIE KING, and Acala MAXXA, or a progeny of any thereof.
